(19) United States
(12) Patent Application Publication
(10) Pub. No.: US 2024/0188832 A1
(43) Pub. Date: Jun. 13, 2024

(54) MONITORING PHYSIOLOGIC PARAMETERS IN HEALTH AND DISEASE USING LIDAR

(71) Applicant: Mayo Foundation for Medical Education and Research, Rochester, MN (US)

(72) Inventors: David Giardi, Jacksonville Beach, FL (US); Clifton R. Haider, Rochester, MN (US); Antonio J. Forte, Jacksonville Beach, FL (US); Rickey E. Carter, Ponte Vedra, FL (US); Christopher J. McLeod, Ponte Vedra Beach, FL (US); Charles J. Bruce, Ponte Vedra Beach, FL (US)

(21) Appl. No.: 18/286,693

(22) PCT Filed: Apr. 13, 2022
(86) PCT No.: PCT/US2022/024537

§ 371 Date: Oct. 12, 2023

Related U.S. Application Data
(60) Provisional application No. 63/174,161, filed on Apr. 13, 2021.

(51) Int. Cl. A61B 5/00; G01S 7/48; G01S 7/481; G01S 17/00; G01S 17/89

(52) U.S. Cl. CPC A61B 5/0082; A61B 5/444; A61B 5/445; G01S 7/4802; G01S 7/4811; G01S 17/003; A61B 2503/06; A61B 2503/40; A61B 2562/0233; G01S 17/89

(57) ABSTRACT

An example system includes an annular support structure defining an aperture, a plurality of light emitters secured to the annular support structure, a plurality of light sensors secured to the annular support structure, and a computer system communicatively coupled to the light emitters and the light sensors. The light emitters are configured to emit a plurality of photons into the aperture. The light sensors are configured to obtain measurement data regarding one or more of the photons reflecting and/or scattering from a subject disposed within the aperture. The computer system is configured to determine one or more properties of the subject based on the measurement data.

### CROSS-REFERENCE TO RELATED APPLICATIONS

This application claims the benefit of U.S. Provisional Application Ser. No. 63/174,161, filed Apr. 13, 2021. The disclosure of the prior application is considered part of (and is incorporated by reference in) the disclosure of this application.

### TECHNICAL FIELD

This disclosure relates to light detection and ranging (LiDAR).

### BACKGROUND

Light detection and ranging (LiDAR) is a technique for determining ranges (e.g., variable distance) by transmitting light at an object using a transmitter, and measuring the time for reflected light to return to a light sensor.

### SUMMARY

In an aspect, a system includes an annular support structure defining an aperture, a plurality of light emitters secured to the annular support structure, a plurality of light sensors secured to the annular support structure, and a computer system communicatively coupled to the light emitters and the light sensors. The light emitters are configured to emit a plurality of photons into the aperture. The light sensors are configured to obtain measurement data regarding one or more of the photons reflecting and/or scattering from a subject disposed within the aperture. The computer system is configured to determine one or more properties of the subject based on the measurement data.

Implementations of this aspect can include one or more of the following features.

In some implementations, the measurement data can include a data record indicating a length of time between an emission of at least some of the photons by one or more of the light emitters and an arrival of those photons at one or more of the light sensors.

In some implementations, the light emitters and the light sensors can alternate along a circumference of the annular support structure.

In some implementations, the annular support structure can have a tubular shape, and the light emitters and light sensors can be re distributed along a length of the annular support structure.

In some implementations, the light emitters can be configured to emit a first subset of the photons according to a first wavelength of light, and emit a second subset of the photons according to a second wavelength of light different from the first wavelength of light.

In some implementations, the annular support structure can be configured to encircle the subject.

In some implementations, the subject can be a portion of a human body.

In some implementations, the portion of the human body can include an arm of the human body.

In some implementations, the computer system can be configured to determine a morphology of skin along the portion of the human body based on the measurement data.

In some implementations, the computer system can be configured to determine a condition of a wound of the portion of the human body based on the measurement data.

In some implementations, the computer system can be configured to determine a health condition of the subject based on the measurement data.

In some implementations, the health condition can be at least one of a rash, carcinoma, lymphedema, lymphadenitis, or leucoplakia.

In some implementations, the subject can be an animal.

In some implementations, the subject can be an adult human.

In some implementations, the subject can be a human child.

In some implementations, the light emitters can be configured to emit the plurality of photons into the aperture continuously during a period of time. Further, the light sensors can be configured to obtain the measurement data continuously during the period of time. Further, the computer system can be configured to determine the one or more properties of the subject continuously over the period of time.

The details of one or more embodiments are set forth in the accompanying drawings and the description below. Other features and advantages will be apparent from the description and drawings, and from the claims.

### DESCRIPTION OF DRAWINGS

FIG. 1 is a diagram of a system for obtaining information regarding a subject using light detection and ranging (LiDAR).

FIGS. 2A and 2B are diagrams of an example LiDAR system.

FIG. 2C is a diagram of another example LiDAR system.

FIG. 3A shows a first histogram representing an amount of light that is emitted over time, and a second histogram representing the amount of light that is detected over time by an example LiDAR system.

FIG. 3B shows plots of times of flights of two wavelengths of light emitted and detected by an example LiDAR system.

FIG. 4 is a diagram of an example system for synchronizing the emission and transmission of light.

FIG. 5 is a diagram of an example computer system.

Like reference symbols in the various drawings indicate like elements.

### DETAILED DESCRIPTION

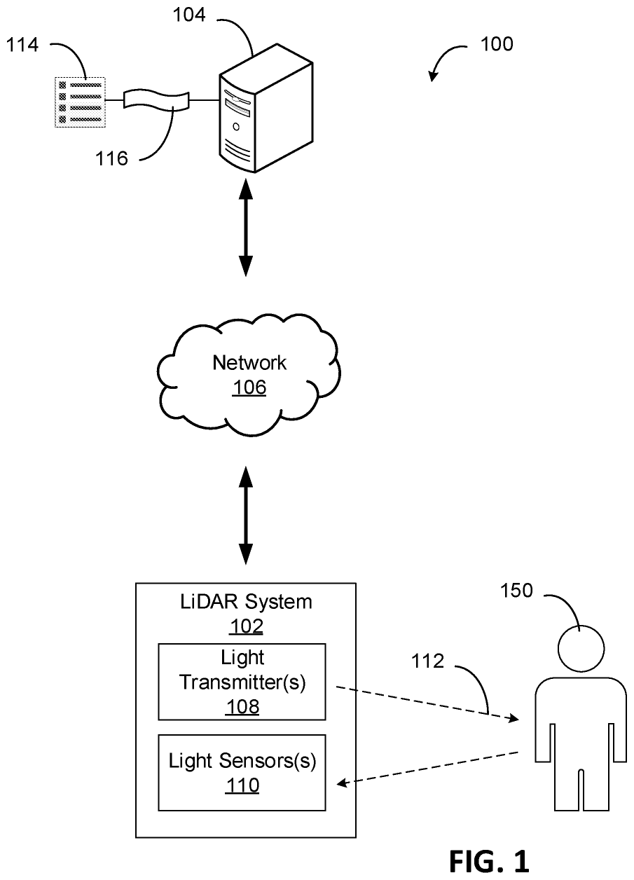

FIG. 1 shows an example system 100 for obtaining information regarding a subject 150 using light detection and ranging (LiDAR). The system 100 includes a LiDAR system 102, a computer system 104 for controlling the operation of the LiDAR system 102, and a network 106 communicatively coupling the LiDAR system 102 to the computer system 104.

The LiDAR system 102 includes one or more light transmitters 108 and one or more light sensors 110. During an example operation of the LiDAR system 102, the light transmitter(s) 108 transmit light 112 towards the subject 150, and detects at least some of the light 112 returning to the LiDAR system 102 using the light sensor(s) 110 (e.g., due to the reflection and/or scattering of the light by the subject 150).

Further, the LiDAR system 102 can measure the time of flight of the light 112 from the light transmitter(s) 108, to the subject 150, and to the light sensor(s) 110 (e.g., the length of time between the time at which the light 112 is transmitted by the light transmitter(s) 108 and the time at which the light 112 is detected by the light sensor(s) 110). This time of flight can be used to determine characteristics of the subject 150. For example, as light propagates through an environment according to a known velocity, the time of flight is proportional to the length of the path of light propagation (i) from the light transmitter(s) 108 to the location on the subject 150 from which the light reflects and/or scatters, and (ii) from that location on the subject 150 to the light sensor(s) 110. Accordingly, a longer time of flight can indicate that the location on the subject 150 is further away from the light transmitter(s) 108 and/or the light sensor(s) 110, whereas a shorter longer time of flight can indicate that the location on the subject 150 is closer to the light transmitter(s) 108 and/or the light sensor(s) 110.

In some implementations, the LiDAR system 102 can use the light transmitter(s) 108 to transmit pulses of light according to a particular spatial pattern, such that pulses of light impinge on different locations of the subject 150. Based on the time of flight of each of these pulses (e.g., as detected using the light sensor(s) 110), the LiDAR system 102 can determine the location of each of the locations of the subject 150 relative to the light transmitter(s) 108 and/or the light sensor(s) 110. In some implementations, the LiDAR system 102 can “scan” the surface of the subject 150 (e.g., by transmitting pulses of light at different locations on the surface of the subject 150), and based on the light that reflects and/or scatters from the subject, generate a three-dimensional point cloud or model representing the surface of the subject 150.

The light transmitter(s) 108 can include any component for transmitting pulses of light towards the subject 150. In some implementations, the light transmitter(s) 108 can include one or more lasers configured to emit light (e.g., one or more laser beams) towards the subject 150 through a process of optical amplification based on the stimulated emission of electromagnetic radiation. In some implementations, the light transmitter(s) 108 can include one more light-emitting diodes (LEDs).

In some implementations, the light transmitter(s) 108 can emit light according to different wavelengths (e.g., ultraviolet, visible, and/or near-infrared light). In some implementations, the light transmitter(s) 108 can modulate the phase of light according to a radiofrequency of the emitted electromagnetic waves.

In some implementations, the light transmitter(s) 108 can include a flash LiDAR system (e.g., which illuminates a field of view by generating a wide diverging, single pulse laser beam, and detects light using a one-dimensional or two-dimensional sensor array, instead of using a single-pixel sensor). In some implementations, the light transmitter(s) 108 can include a phased-array emitter (e.g., a microscopic array of individual antennas that can redirect a signal toward a specific direction, such that the LiDAR system need not be mechanically re-oriented). In some implementations, the light transmitter(s) 108 can include a microelectromechanical mirror (MEMS) (e.g., a single laser that is reflected by a single mirror that spins at a rapid rate to scan a one-dimensional region of interest, or two mirrors to enable a scan of a two-dimensional region of interest).

In some implementations, the light transmitter(s) 108 can emit light according to a narrow range of angles (e.g., a point or beam light source). In some implementations, the light transmitter(s) 108 can emit light according to a range of angles. The LiDAR system 102 can include any number of light transmitter(s) 108 (e.g., one, two, three, four, or more).

The light sensor(s) 110 can include any component for detecting light reflecting and/or scattering from the subject 150. In some implementations, the light sensor(s) 110 can include one or more photodetectors, such as solid state photodetectors (e.g., silicon avalanche photodiodes) or photomultipliers). In some implementations, the light sensor(s) 110 can detect light received from a narrow range of angles (e.g., a point detector). In some implementations, the light sensor(s) 110 can detect light received from a wider range of angles (e.g., an omnidirectional detector). The LiDAR system 102 can include any number of light sensors(s) 110 (e.g., one, two, three, four, or more).

In some implementations, the light sensor(s) 110 can include one or more complementary metal-oxide-semiconductor (CMOS) shutters. For example, a CMOS shutter can include an array of high-speed detectors and modulation sensitive detectors, such as those built on a single chip. Each pixel can perform local processing, such as demodulation or gating at high speed, and down-convert the signals to particular video rate. This technique enables the simultaneous acquisition of multiple of pixels/channels (e.g., thousands of pixels/channels).

In some implementations, the light sensor(s) 110 can detect light according to incoherent or direct energy detection (e.g., whereby a sensor measures amplitude and position of the incident light). In some implementations, the light sensor(s) 110 can detect light according coherent or indirect energy detection (e.g., whereby a sensor detects Doppler shifts or changes in the phase of light, such as based on the principle of optical heterodyne detection). In some implementations, coherent or indirect energy detection may be particularly advantageous, as it is more sensitive, requires less power, and/or is safer for the human eye than incoherent or direct energy detection.

In some implementations, the orientation of the light transmitter(s) 108 and/or light sensor(s) 110 can be fixed relative to a support structure or frame of the LiDAR system 102. In some implementations, the orientation of the light transmitter(s) 108 and/or light sensor(s) 110 can be adjustable relative to a support structure or frame of the LiDAR system 102 (e.g., via one or more motors). In some implementations, the orientation of the light transmitter(s) 108 and/or light sensor(s) 110 can be adjusted manually (e.g., by a user). In some implementations, the orientation of the light transmitter(s) 108 and/or light sensor(s) 110 can be adjusted automatically by the system 100. For example, the system 100 can include a camera to analyze a field of view, recognition software and/or hardware to define a region of interest based on the output of the camera, and a control system to drive one or more motors to adjust the orientation of the light transmitter(s) 108 and/or light sensor(s) 110.

The LiDAR system 102 can obtain measurements according to different fields of view or regions of interest (ROIs). As an example, the LiDAR system 102 can obtain measurements according to a point ROI (e.g., a point), a one-dimensional ROI (e.g., a line), a two-dimensional ROI (e.g., an area), or a three-dimensional ROI (e.g., a volume).

The computer system 104 is communicatively coupled to the LiDAR system 102 via the network 106, and is configured to control the operation of the LiDAR system 102 and/or to process data generated by the LiDAR system 102. For example, the computer system 104 can instruct the LiDAR system 102 to transmit light 112 towards the subject 150 according to a particular spatial and/or temporal pattern. As another example, the computer system 104 can receive light measurements obtained by the system 102, and generate one or more point clouds or models based on the measurements.

The computer systems 104 can include one or more electronic devices that are configured to present, process, transmit, and/or receive data. Examples of the computer system 104 include computers (such as desktop computers, notebook computers, server systems, etc.), mobile computing devices (such as cellular phones, smartphones, tablets, personal data assistants, notebook computers), and other devices capable of presenting, processing, transmitting, and/or receiving data. The computer system 104 can include devices that operate using one or more operating system (e.g., Microsoft Windows, Apple macOS, Linux, Unix, Google Android, Apple iOS, etc.) and/or architectures (e.g., x86, PowerPC, ARM, etc.) In some implementations, the computer system 104 need not be located locally with respect to the rest of the system 100, and the computer system 104 and can be located in one or more remote physical locations.

In FIG. 1, the computer system 104 is illustrated as a single component. However, in practice, the computer system 104 can be implemented on one or more computing devices (e.g., each computing device including at least one processor such as a microprocessor or microcontroller).

The computer system 104 includes a user interface 114. Users interact with the user interface 144 to view data (e.g., data on the computer system 104 and/or the LiDAR system 102). Users also interact with the user interface 114 to issue commands 116. The commands 116 can be, for example, any user instruction to the computer system 104 and/or the LiDAR system 102. In some implementations, a user can install a software application onto the computer system 104 to facilitate performance of these tasks.

The network 106 can be any communications network through which data can be transferred and shared. For example, the network 106 can be a local area network (LAN) or a wide-area network (WAN), such as the Internet. The network 106 can be implemented using various networking interfaces, for instance wireless networking interfaces (e.g., Wi-Fi, Bluetooth, or infrared) or wired networking interfaces (e.g., Ethernet or serial connection). The network 106 also can include combinations of more than one network, and can be implemented using one or more networking interfaces.

In some implementations, the system 100 can be operated according to a time-correlated photon counting mode, whereby the system 100 generates raw photon counts in time. When operating in the raw data mode, the system 100 can gather additional information about a particular ROI, including absorption and scatter coefficients. This enables the system 100 to perform inverse image reconstruction based on the emission of light by the light transmitter(s) 108 and the measurement of light by the light sensors(s) 110.

In some implementations, the system 100 can be used to generate one or more morphological image of the subject. As an example, the images can include visual representations of the subject's bone, tendon, muscle, and other structures. Further, multiple images can be generated over a span of time to show the function of the structures over time. In some implementations, images can be generated in real-time or substantially real-time.

In some implementations, when operating in the raw mode with a finite pulse of light and under the conditions of pure specular reflection, the system 100 can generate an equally shaped pulse with a delay in arrival proportional to the distance the photons traveled. Alternatively, when the same pulse is coupled through a turbid media, the light pulse would arrive delayed and spread through time in relation to the absorption, scatter, and anisotropy coefficients, as well as the delay being greater than the specular delay.

In some implementations, the LiDAR system 102 can be portable, such that it can be readily carried, moved, and/or deployed by a user without assistance. For example, the LiDAR system 102 can be a handheld device.

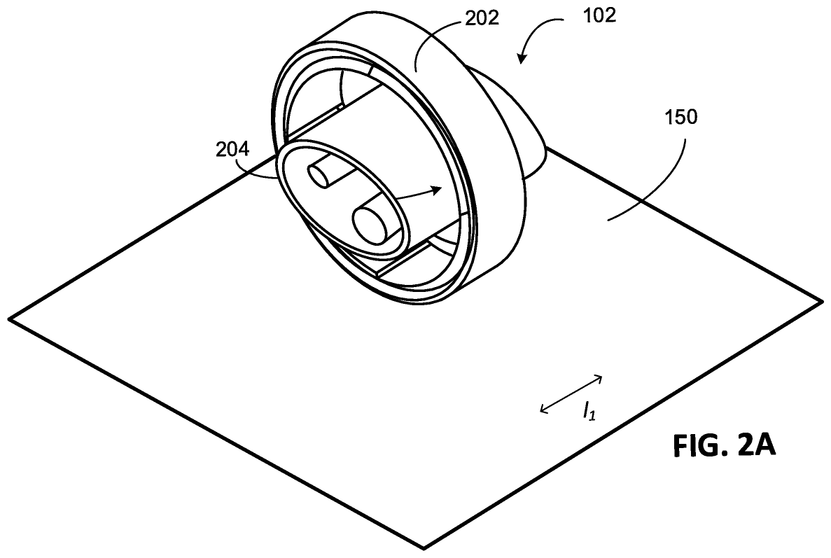

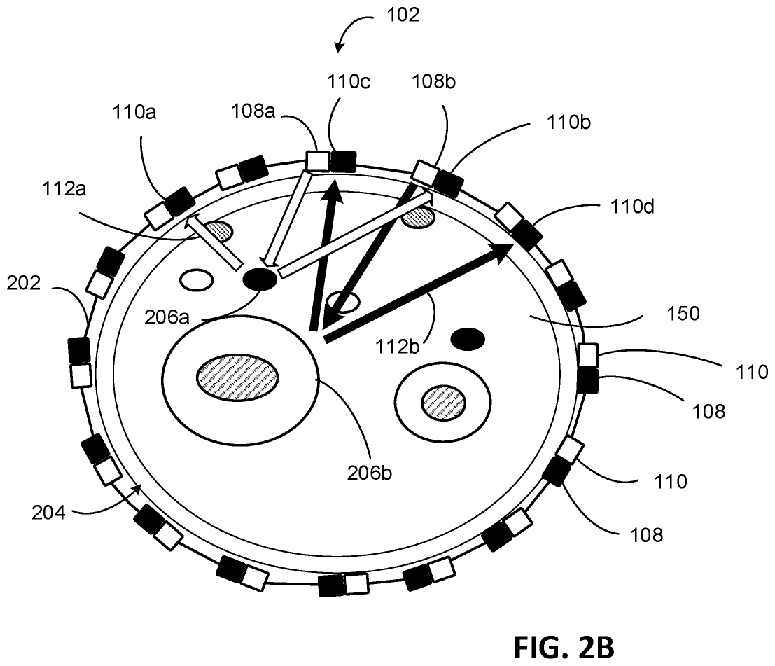

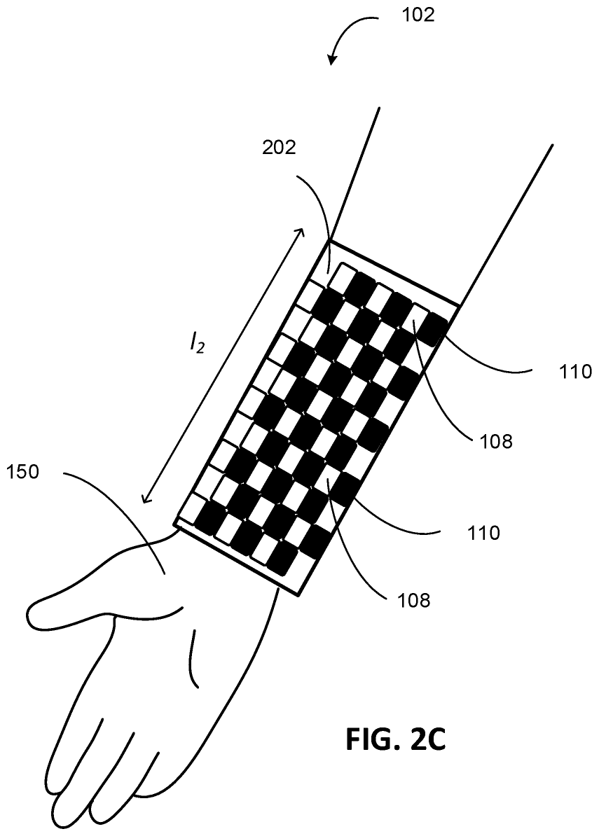

In some implementations, the LiDAR system 102 can be configured to encircle at least a portion of the subject 150, and obtain information regarding that portion of the subject 150. A LiDAR system 102 having this configuration is shown in FIGS. 2A and 2B.

As shown in FIGS. 2A and 2B, the LiDAR system 102 includes an annular support structure 202 defining an aperture 204, and several light transmitters 108 and light sensors 110 secured to the annular support structure 202 and oriented towards the aperture 204.

During an example operation of the LiDAR system 102, the annular support structure 202 is positioned such that it encircles a portion of the subject 150. As an example, the annular support structure 202 can be positioned such that it encircles a subject's arm, leg, torso, head, hand, foot, finger, toe, or any other body part of the subject 150. Further, the LiDAR system 102 transmits light 112 towards the portion of the subject 150 using the light transmitters 108, and detects at least some of the light 112 the light sensors 110.

As shown in FIG. 2B, the propagation paths of the light 112 will vary, depending on the properties of the subject 150 and/or the locations of various structures of the subject 150 relative to the light transmitters 108 and the light sensors 110. Accordingly, the time of flight of the light 112 will also vary. These variations in the time of light of the 112 can be used to obtain information regarding the subject 150, such as the composition of the subject 150 and/or the locations of various structures of the subject 150 relative to the light transmitters 108 and the light sensors 110.

For example, as shown in FIG. 2B, the annular support structure 202 can be positioned such that it encircles an arm of the subject 150. The subject's arm includes various structures, such as bones, tendons, arteries, veins, nerves, and lymphatic vessels, among other structures. The propagation paths of the light 112 emitted by the light transmitters 108 will vary, depending on the properties of each of those structures (e.g., the light absorption, reflection, scattering, and/or transmission properties of those structures) and/or locations of each of those structures.

For example, as shown in FIG. 2B, light 112a emitted by a light transmitter 108a may impinge upon a structure 206a (e.g., an artery) and reflect and/or scatter according to a particular pattern. At least some of the reflected and/or scattered light 112a is detected by one or more of the light sensors (e.g., the light sensors 110a and 110b). Based on the time of flight of the detected light 112a, the LiDAR system 102 can determine one or more properties of the structure 206a and/or the location of the structure 206a.

As another example, as shown in FIG. 2B, light 112b emitted by a light transmitter 108b may impinge upon another structure 206b (e.g., bone) and reflect and/or scatter according to a particular pattern. At least some of the reflected and/or scattered light 112b is detected by one or more of the light sensors (e.g., the light sensors 110c and 110d). Based on the time of flight of the detected light 112b, the LiDAR system 102 can determine one or more properties of the structure 206b and/or the location of the structure 206b.

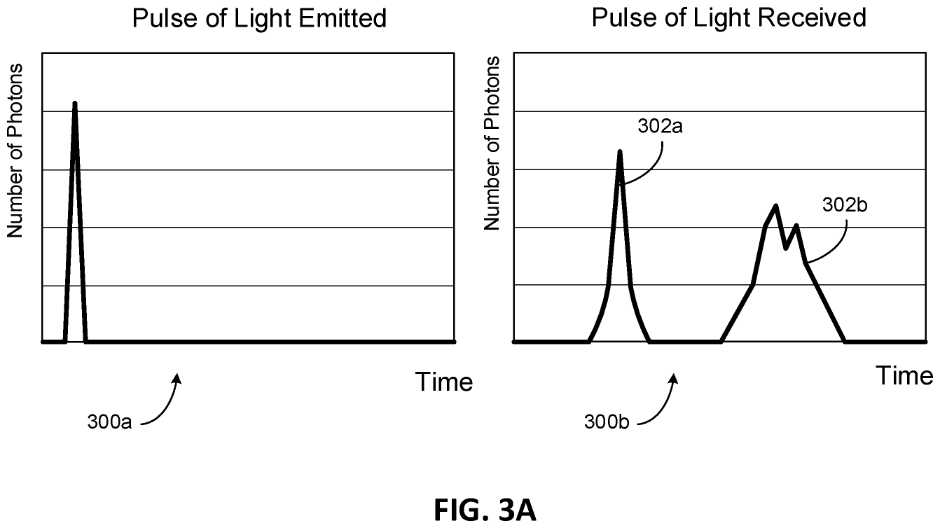

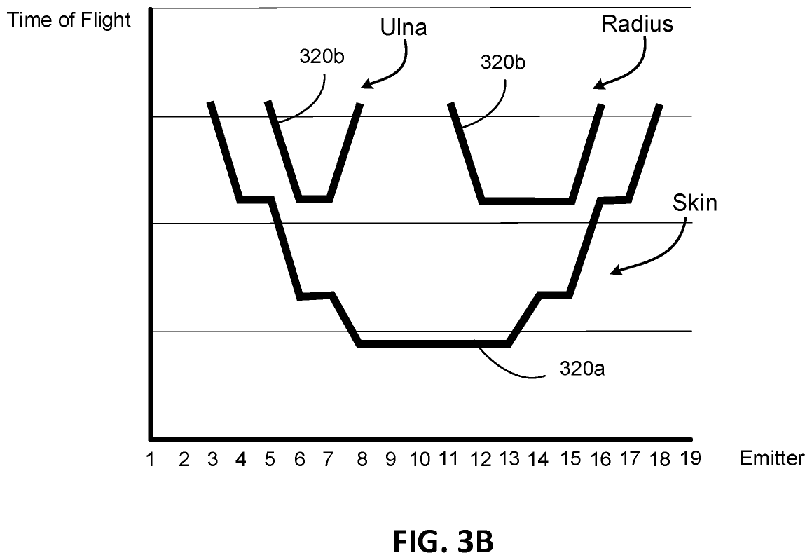

In some implementations, the LiDAR system 102 can emit light during a first time and/or a first time interval, and subsequently detect reflected and/or scattered light during a second time and/or second time interval. In some implementations, the amount of light that is emitted and detected can be presented using histograms. For example, FIG. 3A shows a first histogram 300a representing an amount of light (e.g., a number of photons) that is emitted over time, and a second histogram 300b representing the amount of light (e.g., a number of photons) that is detected over time. In the example shown in FIG. 3A, the emitted light is reflected by the subject into at least two different paths, each having a different path length (and correspondingly, a different time of flight). These paths are indicated by the two distributions 302a and 302b in the histogram 300b.

As shown in FIG. 2B, the light transmitters 108 can be distributed about the periphery of the annular support structure 202 (e.g., along an inner circumference of the annular support structure 202), such that the LiDAR system 102 can emit light according to a range of angles about the subject 150 (e.g., a 360° range). Further, the light sensors 110 also can be distributed about the periphery of the annular support structure 202 (e.g., along an inner circumference of the annular support structure 202), such that the LiDAR system 102 can detect light according to a range of angles about the subject 150 (e.g., a 360° range). In some implementations, the light transmitters 108 and the light sensors 110 can alternate with one another along the periphery of the annular support structure 202.

As shown in FIG. 2A, the annular support structure 202 can have a tubular shape. In practice, the length of the annular supporting structure 202 can vary, depending on the implementation. For example, as shown in FIG. 2A, the annular support structure 202 can have a shorter length l₁, such that it encircles a smaller portion of the subject 150 (e.g., similar to a hoop, ring, or halo). As another example, as shown in FIG. 2C, the annular support structure 202 can have a longer length l₂, such that it encircles a larger portion of the subject 150 (e.g., similar to a sleeve). As shown in FIG. 2C, the light transmitters 108 and/or the light sensors 110 can be distributed along the periphery of the annular support structure 202. For example, the light transmitters 108 and/or the light sensors 110 can alternate with one another along a circumference of the annular support structure 202, and/or along a length of the annular support structure 202.

In some implementations, the LiDAR system 102 can be configured to transmit light according to multiple wavelengths (e.g., in succession, or concurrently), and detect the light that reflects and/or scatters from the subject 150. This can be beneficial, for example, as different structures of the subject 150 may absorb, reflect, scatter, and/or transmit light differently, depending on the wavelength of the light. Accordingly, the LiDAR system 102 can obtain sensor information regarding multiple different wavelengths of light (e.g., according to a particular spectral range) to better distinguish structures from one another.

As an example, FIG. 3B shows a plot 320a of the times of flight of a first wavelength of light λ₁ emitted by the light transmitters 108 towards a subject 150 (e.g., the subject's arm), and a plot 320b of the times of flight of a second wavelength of light λ₂ emitted by the light transmitters 108 towards a subject 150. In this example, the first wavelength of light λ₁ is shorter than the second wavelength of light λ₂. Accordingly, the first wavelength of light λ₁ exhibits a lesser degree of penetration into the tissue of the subject than the second wavelength of light λ₂. Accordingly, the first wavelength of light λ₁ can be used to investigate certain types of tissue that are relatively superficial on the subject's body (e.g., epidermis, derma, and/or hypodermic layers of the body), whereas the second wavelength of light λ₂ can be used to investigate certain other types of tissue that are relatively deeper within the subject's body (e.g., periosteum and bone surfaces).

In some implementations, the LiDAR system 102 can emit and detect light according to a synchronized pattern. For example, according to a first cycle, a first one of the light transmitters can emit light, and subsequently, light can be detected using one or more of the light sensors 110. Further, according to a second cycle, a second one of the light transmitters can emit light, and subsequently, light can be detected using one or more of the light sensors 110. Further, according to a third cycle, a third one of the light transmitters can emit light, and subsequently, light can be detected using one or more of the light sensors 110. These cycles can repeat until light is emitted by some or all of the light transmitters 108 in a sequence, and the corresponding reflected and/or scattered light is detected.

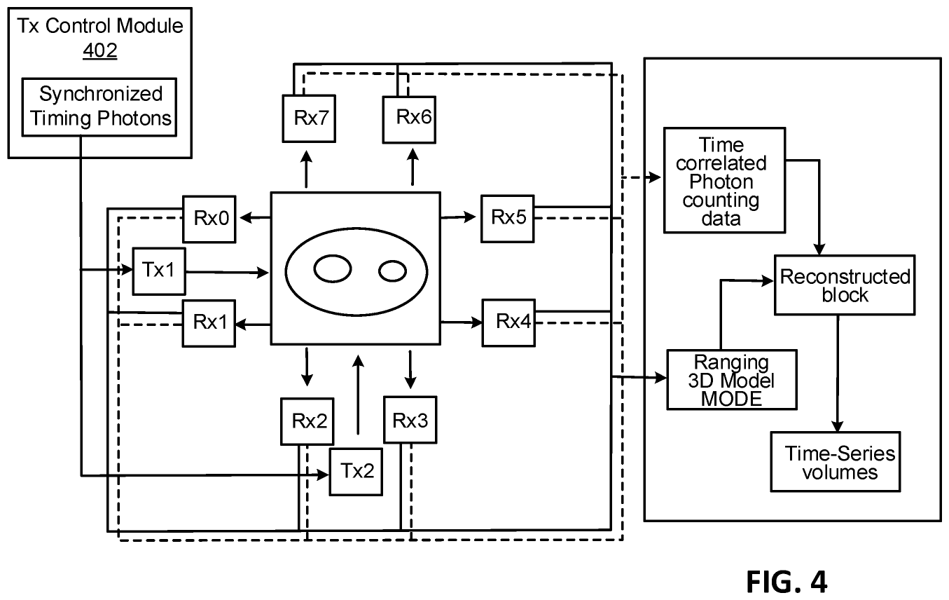

FIG. 4 shows an example system 400 for synchronizing the emission and transmission of light. The system 400 can be included, for example, in the LiDAR system 102. The system 400 includes several light transmitters (labeled as Tx1, . . . , TxN), and several light sensors 110 (labeled as Rx1, . . . , RxN). Further, the system 400 includes a transmission control module 402 that is communicatively coupled to each of the light transmitters, and selectively activates certain ones of the light transmitters according to a particular timing schedule.

Further, the system 400 includes a receiver control module 404 that is communicatively coupled to each of the light sensors, and selectively activates certain ones of the light sensors according to a particular temporal pattern or timing schedule (e.g., shortly after the activation of each of the light transmitters) to detect light reflecting and/or scattering from the subject. Based on the measurements obtained by the light sensors, the receiver control module 404 can generate time-correlated photon counting data (e.g., a histogram of detected photons over time) and/or a three-dimensional model of the subject. This data can be reconstructed and/or aggregated over time to generate time-series volumetric data regarding the subject.

### Example Applications

The example systems and techniques described herein can be used to monitor the health of a subject (e.g., a human patient).

As an example, the systems and techniques described herein can be used to generate an accurate dynamic and/or static reproduction of a surface ROI of a subject (e.g., a three-dimensional model, point cloud, or other mathematical representation), as well as time-correlated photon counting arrivals of photons. The model, point cloud, and/or mathematical representation can be analyzed (e.g., in real-time or substantially real-time, or in a post-processing stage) to collect information and support a diagnosis, screening, and/or therapy process. Further, the use of multiple wavelengths can provide multi-spectral, wavelength-specific, information about the composition of the ROI. The addition of a transmitter-to-many-receiver pairings can also increase the capture of photons from by the system, and improve its sensitivity. Further, these system and techniques can be used to obtain information regarding a subject quickly and in a non-invasive manner. Further, these system and techniques can be used in a wide array of heterogeneous settings, such as during an outpatient visit, during a surgery, and/or in a domestic setting using a smartphone.

As an example, the systems and techniques described herein can be used in a clinical setting to image a subject's tissues (e.g., bone, vessels, nerves, tendons, and muscles) and tissue function (e.g., real-time or substantially real time imaging of tendon movement and of muscle contraction). Further, the systems and techniques described herein do not require ionizing radiation. Accordingly, the systems and techniques described herein can be used for patients that would otherwise be at risk of undergoing ionized imaging studies, such as children and pregnant women.

As another example, in a procedural or interventional radiology setting (e.g., during a placement of a pacemaker lead or a placement of a stent), the systems and techniques described herein can enable continuous, radiation-free, real-time (or substantially real-time) imaging, and can facilitate the accurate placement of devices and catheters.

As another example, for hand surgeries, the use of a LiDAR system having an annular shape enables assessment of tendon gliding, diagnosis of bone fracture, diagnosis of nerve, or vessel damage, without the use of large diagnostic machines, devices that expose patients to ratio, and/or devices that cannot be used to image patients having metal in their bodies (e.g., magnetic resonance imaging system). For instance, for patients with phalangeal bone fracture, a LiDAR system can be used to determine a static morphology of the bone and to identify the fracture. As another example, for patients who present with a trigger finger, a LiDAR system can be used to determine the static morphology (e.g., to show an intact tendon) and to perform a dynamic functional study (e.g., to show that the gliding of the tendon is impaired as it becomes entrapped within the A1 pulley). As another example, both static and dynamic studies can be performed to diagnose a tendon rupture or laceration. For instance, a static morphology can show a disrupted tendon, and a dynamic functional study can show the proximal end of the tendon retracting and the discontinuity between the proximal and distal ends of the tendon.

As another example, a LiDAR system can be used to perform intraoperative imaging during a surgical procedure. For example, a LiDAR system can be used to provide information regarding the location and condition of one or more bones, muscle, tendons, ligaments, etc. of the subject. Further, the LiDAR system can be used to guide the insertion and fix screws or other impacts in the subject. In some implementations, a LiDAR system can provide information in real-time or substantially real time during the surgical procedure. In some implementations, a LiDAR system can be used in place of other imaging systems (e.g., a fluoroscopy imaging system).

As another example, the systems and techniques described herein can be used to characterize physiologic or pathologic skin and/or body surfaces. For example, the systems and techniques described herein can be used to scan the surface of a subject's body (e.g., skin, cutaneous annexes, accessible mucosae, digestive tract, respiratory tract, genitourinary tract, lymphatic ducts, etc.) and generate can a three-dimensional morphological model. This model can be used to conduct further analysis, such as to conduct screening (e.g., nevus control, etc.), diagnose and/or monitor pathological signs or conditions (e.g., wound healing, rash, carcinoma, lymphedema, lymphadenitis due to infections, leucoplakia, etc.).

As another example, the systems and techniques described herein can be used to detect dynamic skin and/or body surface conformation as an indicator of physiological or pathological signs due to sub-skin/surface tissues and structures (e.g., carotid pulse, jugular vein pulse, radial artery pulse, joint inflammation, fractures, adipose tissue quantity, hematomas, hernias, infections, JVD, caput medusae in cirrhosis, etc.). The systems and techniques described herein can be used could be used for vital signs detection (e.g., the use of the sensor to detect chest wall movement as an indicator of respiration, such as a wall-mounted sensor over the bed) to detect sleep-disordered breathing/decompensated heart failure. Further, the systems and techniques described herein can be used to analyze the skin dynamic of a subject due to the underneath presence of arterial and/or venous vessels.

As another example, the systems and techniques described herein can be used to characterize physiologic or pathologic subsurface structures using of multiple LiDAR sensors. For example, a subject can be evaluated from different angles of incidence. Further, a subject can be evaluated on an analysis of different absorptions and reflection/backscattering indexes of different characteristics (e.g., wavelengths, phase, etc.) interacting with tissues at variable depths.

In some implementations, the systems and techniques described herein can be used to monitor the health of a subject (e.g., a human patient) in lieu of using a computerized tomography (CT) imaging system and/or an X-ray imaging system. This can be beneficial, for example, in reducing the subject's exposure to ionizing radiation. Nevertheless, in some implementations, the systems and techniques described herein can be used to monitor the health of a subject in conjunction with a CT imaging system and/or an X-ray imaging system (e.g., to provide additional information regarding the subject to supplement that provided by the other imaging systems).

In some implementations, the systems and techniques described herein can be used to monitor the health of a subject (e.g., a human patient) in lieu of using a magnetic resource imaging (MRI) system. This can be beneficial, for example, in obtaining information regarding a subject that cannot be safely examined using an MRI system (e.g., a subject having metal implants). Nevertheless, in some implementations, the systems and techniques described herein can be used to monitor the health of a subject in conjunction with an MRI system (e.g., to provide additional information regarding the subject to supplement that provided by the MRI system).

In some implementations, the systems and techniques described herein can be operated on a continuous basis (e.g., to provide information regarding the subject on a continuous basis). Further, the systems and techniques described herein can be automated, such that they do not rely on input or commands from the subject. This can be beneficial, for example, in allowing a physician or caretaker to continuously monitor the health of a subject, without requiring that the subject manually operate the system and/or manually perform the techniques described herein.

In some implementations, the systems and techniques described herein can be operated to obtain information at one or more discrete points in time (e.g., to provide information regarding the subject at specific points in time).

In general, the systems and techniques described herein can be used to monitor the health of a subject of any age. As an example, the systems and techniques described herein can be used to monitor the health of adult subjects (e.g., humans who are eighteen years or older). As an example, the systems and techniques described herein can be used to monitor the health of subjects who are children (e.g., humans who are under eighteen years old), such as part of a pediatric imaging protocol. Use of the systems and techniques described herein may be particularly advantageous to monitor the health of subjects who are children, as the systems and technique described herein do not rely on the use of ionizing radiation.

In general, the systems and techniques described herein also can be used to monitor the health of an animal subject (e.g., veterinary imaging). Animals can include vertebrates (e.g., fishes, amphibians, reptiles, birds, mammals, etc.) and invertebrates (e.g., nematodes, arthropods, mollusks, etc.). Example animals include dogs, cats, horses, cows, pigs, ferrets, weasels, birds, snakes, fish, rabbits, mice, rats, turtles, or any other animal.

### Example Systems

Some implementations of subject matter and operations described in this specification can be implemented in digital electronic circuitry, or in computer software, firmware, or hardware, including the structures disclosed in this specification and their structural equivalents, or in combinations of one or more of them. For example, in some implementations, the LiDAR system 102 and/or the computer system 104 can be implemented using digital electronic circuitry, or in computer software, firmware, or hardware, or in combinations of one or more of them.

Some implementations described in this specification can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and optical disks (e.g., CD-ROM disks, DVD-ROM disks, Blu-ray disks, etc.). The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

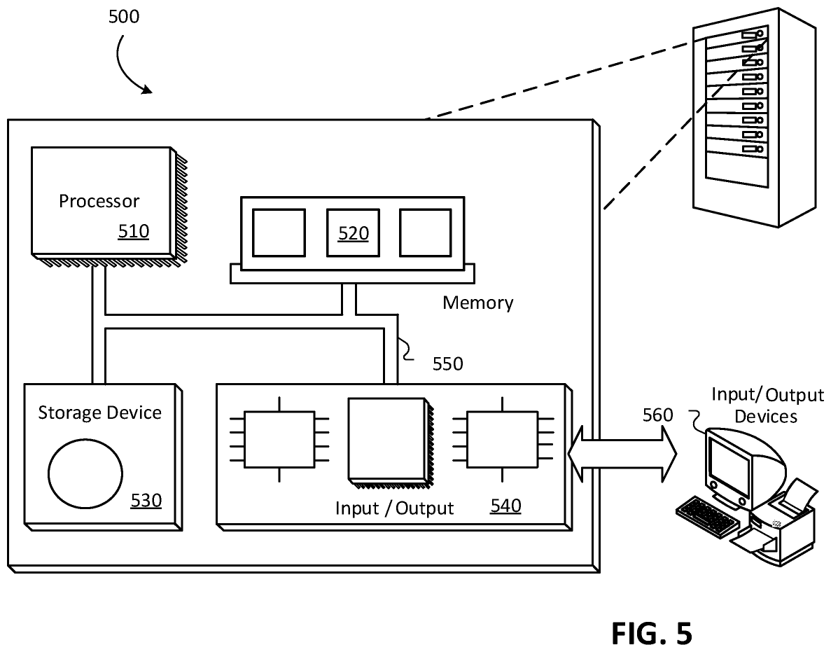

FIG. 5 shows an example computer system 500 that includes a processor 500, a memory 520, a storage device 530 and an input/output device 540. Each of the components 510, 520, 530 and 540 can be interconnected, for example, by a system bus 550. The processor 510 is capable of processing instructions for execution within the system 500. In some implementations, the processor 510 is a single-threaded processor, a multi-threaded processor, or another type of processor. The processor 510 is capable of processing instructions stored in the memory 520 or on the storage device 530. The memory 520 and the storage device 530 can store information within the system 500.

The input/output device 540 provides input/output operations for the system 500. In some implementations, the input/output device 540 can include one or more of a network interface device, e.g., an Ethernet card, a serial communication device, e.g., an RS-232 port, and/or a wireless interface device, e.g., an 802.11 card, a 3G wireless modem, a 4G wireless modem, a 5G wireless modem, etc. In some implementations, the input/output device can include driver devices configured to receive input data and send output data to other input/output devices, e.g., keyboard, printer and display devices 560. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of implementations have been described. Nevertheless, it will be understood that various modifications may be made without departing from the spirit and scope of the invention. Accordingly, other implementations are within the scope of the following claims.

What is claimed is:

1. A system comprising:
an annular support structure defining an aperture;
a plurality of light emitters secured to the annular support structure;
a plurality of light sensors secured to the annular support structure; and
a computer system communicatively coupled to the light emitters and the light sensors,
wherein the light emitters are configured to emit a plurality of photons into the aperture,
wherein the light sensors are configured to obtain measurement data regarding one or more of the photons reflecting and/or scattering from a subject disposed within the aperture, and
wherein the computer system is configured to determine one or more properties of the subject based on the measurement data.

2. The system of claim 1, wherein the measurement data comprises a data record indicating a length of time between an emission of at least some of the photons by one or more of the light emitters and an arrival of those photons at one or more of the light sensors.

3. The system of claim 1, wherein the light emitters and the light sensors alternate along a circumference of the annular support structure.

4. The system of claim 1, wherein the annular support structure has a tubular shape, and wherein the light emitters and light sensors are distributed along a length of the annular support structure.

5. The system of claim 1, wherein the light emitters are configured to:
emit a first subset of the photons according to a first wavelength of light, and
emit a second subset of the photons according to a second wavelength of light different from the first wavelength of light.

6. The system of claim 1, wherein the annular support structure is configured to encircle the subject.

7. The system of claim 6, wherein the subject is a portion of a human body.

8. The system of claim 7, wherein the portion of the human body comprises an arm of the human body.

9. The system of claim 7, wherein the computer system is configured to determine a morphology of skin along the portion of the human body based on the measurement data.

10. The system of claim 7, wherein the computer system is configured to determine a condition of a wound of the portion of the human body based on the measurement data.

11. The system of claim 7, wherein the computer system is configured to determine a health condition of the subject based on the measurement data.

12. The system of claim 11, wherein the health condition is at least one of a rash, carcinoma, lymphedema, lymphadenitis, or leucoplakia.

13. The system of claim 1, wherein the subject can be an animal.

14. The system of claim 1, wherein the subject is an adult human.

15. The system of claim 1, wherein the subject is a human child.

16. The system of claim 1, wherein the light emitters are configured to emit the plurality of photons into the aperture continuously during a period of time,
wherein the light sensors are configured to obtain the measurement data continuously during the period of time, and
wherein the computer system is configured to determine the one or more properties of the subject continuously over the period of time.
